(19) United States
(12) Patent Application Publication
(10) Pub. No.: US 2004/0105852 A1
(43) Pub. Date: Jun. 3, 2004

(54) Parenteral composition for treating bacterial illnesses

(76) Inventors: Vincent Pischetti, West Hempstead, NY (US); Lawrence Loomis, Columbia, MD (US)

Correspondence Address: KAREN BAILEY YOUNG/, HORIZONS DIAGNOSTICS CORPORATION, 9110 Red Branch Road, Columbia, MD, 21046, US

(21) Appl. No.: 10/720,265

(22) Filed: Nov. 25, 2003

Related U.S. Application Data
(63) Continuation of application No. 09/752,731, filed on Jan. 3, 2001.
(63) Continuation-in-part of application No. 09/482,992, filed on Jan. 14, 2000, now Pat. No. 6,264,945.
Application No. 09/482,992 is a continuation-in-part of application No. 09/395,636, filed on Sep. 14, 1999, now Pat. No. 6,056,954.

(51) Int. Cl. A61K 38/47; A61K 38/48

(52) U.S. Cl. 424/94.61; 424/94.63

(57) ABSTRACT

The present invention discloses a method and composition for the treatment of bacterial infections by the parenteral introduction of an effective amount of at least one lytic enzyme produced by a bacteria infected with a bacteriophage specific for said bacteria wherein the lytic enzyme is selected from the group consisting of shuffled lytic enzymes, chimeric lytic enzymes, holin enzymes, and combinations thereof, wherein said lytic enzyme is in an appropriate carrier for delivering the lytic enzyme into a patient. The injection can be done intramuscularly, subcutaneously, or intravenously.

The following application is a continuation in part of U.S. patent application Ser. No. 09/482,992, filed Jan. 14, 2000, which is a continuation in part of U.S. patent application Ser. No. 09/395,636, filed Sep. 14, 1999.

### DESCRIPTION

### Background of the Invention

1. Field of the Invention

The present invention discloses a method and composition for the treatment of bacterial infections by the parenteral introduction of holin lytic enzymes, shuffled lytic enzymes, and/or chimeric lytic enzymes, blended with an appropriate carrier into a patient. The injection can be done intramuscularly, subcutaneously, or intravenously.

2. Description of the Prior Art

In the past, antibiotics have been used to treat various infections. The work of Selman Waksman in the introduction and production of Streptomycetes, Dr. Fleming's discovery of penicillin, and the work of numerous others in the field of antibiotics are well known. Over the years, there have been additions and chemical modifications to the “basic” antibiotics in attempts to make them more powerful, or to treat people allergic to these antibiotics.

Others have found new uses for these antibiotics. U.S. Pat. No. 5,260,292 (Robinson et al.) discloses the topical treatment of acne with aminopenicillins. The method and composition for topically treating acne and acneiform dermal disorders includes applying an amount of an antibiotic, effective for treating acne and acneiform dermal disorders, selected from the group consisting of ampicillin, amoxicillin, other aminopenicillins, and cephalosporins, and derivatives and analogs thereof. U.S. Pat. No. 5,409,917 (Robinson et al.) discloses the topical treatment of acne with cephalosporins.

However, as more antibiotics have been prescribed or used at an ever increasing rate for a variety of illnesses, increasing numbers of bacteria have developed a resistance to antibiotics. Larger doses of stronger antibiotics are now being used to treat ever more resistant strains of bacteria. Consequently, multiple antibiotic resistant bacteria have been developed. The use of more antibiotics and the number of bacteria showing resistance has led to increases in the amount of time that the antibiotics need to be used. Broad, nonspecific antibiotics, some of which have detrimental effects on the patient, are now being used more frequently. Additionally, the number of people showing allergic reactions to antibiotics appears to be increasing.

Consequently, other efforts have been sought to first identify and then kill bacteria.

Attempts have been made to treat bacterial diseases with the use of bacteriophages. U.S. Pat. No. 5,688,501 (Merril, et al.) discloses a method for treating an infectious disease caused by bacteria in an animal with lytic or non-lytic bacteriophages that are specific for particular bacteria.

U.S. Pat. No. 4,957,686 (Norris) discloses a procedure of improved dental hygiene which introduces into the mouth bacteriophages parasitic to bacteria which possess the property of readily adhering to the salivary pellicle.

It is to be noted that the direct introduction of bacteriophages into an animal to prevent or fight diseases has certain drawbacks. Specifically, the bacteria must be in the right growth phase for the phage to attach. Both the bacteria and the phage have to be in the correct and synchronized growth cycles. Additionally, there must be the right number of phages to attach to the bacteria. The phage must also be active enough. The phages are also inhibited by many substances including bacterial debris from the organism it is going to attack. Further complicating the direct use of bacteriophages to treat bacterial infections is the possibility of immunological reactions, rendering the phage nonfunctional. Another problem is the mutation of the receptor on the bacterial surface preventing bacteriophage attachment.

Consequently, others have explored the use of other safer and more effective means to treat and prevent bacterial infections.

U.S. Pat. No. 5,604,109 (Fischetti et al.) relates to the rapid detection of Group A streptococci in clinical specimens, through the enzymatic digestion by a semi-purified Group C streptococcal phage associated lysin enzyme. The present invention is based upon the discovery that phage lytic enzymes specific for bacteria infected with a specific phage can effectively and efficiently break down the cell wall of the bacterium in question. At the same time, the substrate for the enzyme is not present in mammalian tissues, and therefore is nondestructive to mammalian proteins and tissues when present during the digestion of the bacterial cell wall.

U.S. Pat. No. 5,985,271 (Fischetti, et. al.), U.S. Pat. No. 5,997,862 (Fischetti et al.), and U.S. Pat. No. 6,017,528 (Fischetti et al.) disclose the compositions and use of an oral delivery mode, such as a candy, chewing gum, lozenge, troche, tablet, a powder, an aerosol, a liquid or a liquid spray, containing a lysin enzyme produced by group C streptococcal bacteria infected with a C1 bacteriophage for the prophylactic and therapeutic treatment of Streptococcal A throat infections, commonly known as strep throat. This is the lysin enzyme of U.S. Pat. No. 5,604,109

The same general technique used to produce and purify the lysin enzyme in U.S. Pat. No. 5,604,109 may be used to manufacture other lytic enzymes produced by bacteria infected with a bacteriophage specific for that bacteria. Depending on the bacteria, there may be variations in the growth media and conditions.

The use of phage associated lytic enzymes produced by the infection of a bacteria with a bacteria specific phage has numerous advantages for the treatment of diseases. Lytic enzymes have similar characteristics as their complementary phage in that both are targeted for specific bacteria and neither, when selected, interferes with the functioning of normal bacterial flora. Also, lytic phages primarily attack cell wall structures which are not affected by plasmid variation. The actions of the lytic enzymes are fast and do not depend on bacterial growth. Additionally, lytic enzymes can be directed to the mucosal lining, where, in residence, they will be able to kill colonizing bacteria.

U.S. Pat. No. 6,056,954 (Fischetti et al.) discloses a method and composition for the prophylactic or therapeutic treatment of bacterial infections, comprising administering an effective amount of at least one lytic enzyme produced by a bacteria infected with a bacteriophage specific for the bacteria to the site of the infection. The lytic enzyme preferably comprises a carrier suitable for delivering the lytic enzyme to the site of the infection. This method and treatment may be used for treating upper respiratory infections, topical infections, vaginal infections, eye infections, ear infections, for parenteral treatment, and for most other bacterial infections.

U.S. Pat. No. 6,056,955 (Fischetti et al.) discloses the topical treatment of streptococcal infections.

The use of phage associated lytic enzymes produced by the infection of a bacteria with a bacteria specific phage has numerous advantages for the treatment of diseases. As the phage are targeted for specific bacteria, the lytic enzymes do not interfere with normal flora. Also, lytic phages primarily attack cell wall structures which are not affected by plasmid variation. The actions of the lytic enzymes are fast and do not depend on bacterial growth.

However, sometimes the bacterial infections, by the time they are treated, have developed into more serious illnesses. For example, dermatological infections such as Staphylococcus aureus and Streptococcal pneumoniae can develop into cellulitis, which, unchecked, can lead to a degradation of the connective tissue, septicemia, and possibly death. Other bacterial infections can also evolve into deep tissue infections. Other infections by other bacteria, not necessarily dermatological by nature, can infect and localize in certain tissues of the body, making the infections difficult to treat.

U.S. application Ser. No. 09/482,992 discloses a method and composition for the treatment of bacterial infections by the parenteral introduction of at least one lytic enzyme produced by a bacteria infected with a bacteriophage specific for that bacteria and an appropriate carrier for delivering the lytic enzyme into a patient. The injection can be done intramuscularly, subcutaneously, or intravenously.

### SUMMARY OF THE INVENTION

The method for obtaining and purifying the lytic enzyme produced by a bacteria infected with the bacteriophage is known in the art. Some recent evidence suggests that the phage enzyme that lyses the streptococcus organism may actually be a bacterial enzyme that is used to construct the cell wall and the phage. While replicating in the bacterium, a phage gene product may cause the upregulation or derepression of a bacterial enzyme for the purpose of releasing the bacteriophage. These bacterial enzymes may be tightly regulated by the bacterial cell and are used by the bacteria for the construction and assembly of the cell wall.

The use of these lytic enzymes for the prophylactic and therapeutic treatment of bacterial diseases, however, has not been explored, except by the inventors of the present invention. The lytic enzymes produced by bacterial phages are specific and effective for killing select bacteria.

It is the current trend in biotechnology to do genomic sequencing of microorganisms in order, in part, to aid in the determination of designing drugs for treatment. This type of modeling is both timely and costly, and may or may not lead to new drug discoveries. The elegance of this invention resides in the fact that phage-induced lytic enzymes have evolved into extremely effective and targeted killing agents of selected bacteria. The primary structure and sequencing of purified enzymes serve as templates from which a variety of chemical procedures (such as shuffling) can be performed to optimize the enzyme's effectiveness.

These phage-induced lytic enzymes are useful in killing a variety of bacterial pathogens including those involved in classical clinical diseases such Streptococcus, Pseudomonas, etc. and in biowarfare agents, such as Bacillus and Yersinia, causing anthrax & plague, respectively, among other uses.

The present invention discloses the extraction and use of a variety of bacterial phage associated holin lytic enzymes, chimeric lytic enzymes, and shuffled lytic enzymes, in addition to lytic enzymes, for increased efficiency for the treatment of a wide variety of illnesses caused by bacterial infections.

The lytic system consists of a holin and at least one peptidoglycan hydrolase, or “lysin,” capable of degrading the bacterial cell wall. Lysins can be endo—N-acetylglucosaminidases or N-acetylmuramidases (lysozymes), which act on the sugar moiety, endopeptidases which act on the peptide cross bridge, or more commonly, a N-acetylmuramoyl-L-alanine amidase, which hydrolyzes the amide bond connecting the sugar and peptide moieties. Typically, the holin is expressed in the late stages of phage infection forming a pore in the cell membrane, allowing the lysin(s) to gain access to the cell wall peptidoglycan resulting in release of progeny phage. Significantly, exogenously added lysin can lyse the cell wall of bacterial cells, producing a phenomenon known as “lysis from without.” However, in the case of gram-negative bacteria which contain an outer membrane, the presence of exogenously added holin in combination with a lytic enzyme may allow more efficient access of the lytic enzyme to the peptidoglycan enabling better lysis.

For definitional purposes, shuffled enzymes are enzymes where more than one sequence of usually more than one particular enzyme has been cleaved in one or more locations, and reconstructed in a specific or random order, increasing their activity or specificity.

Chimeric enzymes are enzymes which are a combination of two or more enzymes having two or more active sites such that the chimeric enzyme can act independently on the same or different molecules. This will allow for potentially treating two or more different bacterial infections at the same time.

Holin enzymes produce holes in the cell membrane. More specifically, holin enzymes form lethal membrane lesions that terminate respiration. Like the lytic enzymes, the holin enzymes are coded for and carried by a phage genome. In fact, it is quite common for the genetic code for the holin enzyme to be found next to or even within the code for the lytic enzyme in the phage. Most holin sequences are short, and overall, hydrophobic in nature, with a highly hydrophilic carboxy-terminal domain. In many cases, the putative holin enzyme is encoded on a different reading frame within the enzymatically active domain of the phage. In other cases, the holin enzyme is encoded on the DNA next to or close to the DNA coding for the phage. The holin enzyme is frequently synthesized during the late stage of phage infection and found in the cytoplasmic membrane where it causes membrane lesions.

More specifically, the sequence of enzymes when purified can be determined by conventional techniques, and rearrangements of primary structures can be achieved by state of the art techniques, such as shuffling, to increase the activity and stability of the enzyme(s). Shuffling also allows for combination enzymes (“chimeric enzymes”) to have more than one activity.

The creation, purification, and isolation of chimeric, shuffled, lytic, and holin enzymes are well known to those skilled in the art. In particular, U.S. Pat. No. 6,132,970 (Stemmer) (incorporated herein by reference) discloses a number of new techniques, and modifications of more established procedures, for the creation of these enzymes. The proposed invention utilizes these techniques and applies them for the enhancement of specifically noted phage associated lytic enzymes. The technique for isolating lysin enzymes found in U.S. Pat. No. 6,056,954 (also incorporated herein by reference) may be applied to other phage associated lytic enzymes. Similarly, other state of the art techniques may be used to isolate lytic enzymes.

In a preferred embodiment of the invention, shuffled enzymes are used to treat bacterial infections, thereby increasing the speed and efficiency with which the bacteria are killed.

Chimeric enzymes may also be used to treat one bacterial infection by cleaving the cell wall in more than one location.

A number of chimeric lytic enzymes have been produced and studied. Gene E-L, a chimeric lysin constructed from bacteriophages phi X174 and MS2 lysis proteins E and L, respectively, was subjected to internal deletions to create a series of new E-L clones with altered lysis or killing properties. The lytic activities of the parental genes E, L, E-L, and the internal truncated forms of E-L were investigated in this study to characterize the different lysis mechanism, based on differences in the architecture of the different membranes spanning domains. Electron microscopy and release of marker enzymes for the cytoplasmic and periplasmic spaces revealed that two different lysis mechanisms can be distinguished depending on penetrating of the proteins of either the inner membrane or the inner and outer membranes of the E. coli. FEMS Microbiol. Lett. 1998 Jul 1, 164(1); 159-67.

In another experiment an active chimeric cell wall lytic enzyme (TSL) has been constructed by fusing the region coding for the N-terminal half of the lactococcal phage Tuc2009 lysin and the region coding for the C-terminal domain of the major pneumococcal autolysin. The chimeric enzyme exhibited a glycosidase activity capable of hydrolysing choline-containing pneumococcal cell walls.

A preferred embodiment of this invention discloses the use of chimeric lytic enzymes to treat two infectious bacteria at the same time, or to cleave the cell wall of a bacteria in two different locations.

Holin enzymes can be grouped into two general classes based on primary structure analysis. Class I holins are usually 95 residues or longer and may have three potential transmembrane domains. Class II holins are usually smaller, at approximately 65-95 residues, and the distribution of charged and hydrophobic residues indicating two TM domains (Young, et al. Trends in Microbiology v. 8, No. 4, March 2000). At least for the phages of gram-positive hosts, however, the dual-component lysis system may not be universal. Although the presence of holins has been shown or suggested for several phages, no genes have yet been found encoding putative holins for all of the phages. Holins have been shown to be present or suggested for among others, lactococcal bacteriophage Tuc2009, lactococcal φLC3, pneumococcal bacteriophage EJ-1, Lactobacillus gasseri bacteriophage φadh, Staphylococcus aureus bacteriophage Twort, Listeria monocytogenes bacteriophages, pneumococcal phage Cp-1, Bacillus subtillis phage φ29, Lactobacillus delbrueckki bacteriophage LL-H lysin, and bacteriophage φ11 of Staphylococcus aureus. (Loessner, et al., Journal of Bacteriology, August 1999, p. 4452-4460).

In another embodiment of the invention, holin enzymes are used in conjunction with the lytic enzymes to accelerate the speed and efficiency at which the bacteria are killed. Holin enzymes may also be in the form of chimeric and/or shuffled enzymes. Holin enzymes may also be used alone in the treatment of bacterial infections

In another embodiment of the invention, the holin enzymes are shuffled holin enzymes or chimeric holin enzymes, in either combination with or independent of the lytic enzymes.

The invention (which incorporates U.S. Pat. No. 5,604,109 in its entirety by reference) uses a lytic enzyme produced by the bacterial organism after being infected with a particular bacteriophage as either a prophylactic treatment for preventing those who have been exposed to others who have the symptoms of an infection from getting sick, or as a therapeutic treatment for those who have already become ill from the infection. The present invention is based upon the discovery that phage lytic enzymes specific for bacteria infected with a specific phage can effectively and efficiently break down the cell wall of the bacterium in question. At the same time, the semipurified enzyme is lacking in proteolytic enzymatic activity and therefore is nondestructive to mammalian proteins and tissues when present during the digestion of the bacterial cell wall. As discussed above, the lytic enzymes may be chimeric, shuffled or “natural,” and may be in combination with at least one holin enzyme, which may also be chimeric, shuffled, or “natural.” It is another object of the invention to apply at least one holin lytic enzyme, at least one chimeric lytic enzyme, at least one shuffled lytic enzyme, or combinations thereof, intravenously, to treat septicemia and general infections.

It is also an object of the invention to inject at least one holin lytic enzyme, at least one chimeric lytic enzyme, at least one shuffled lytic enzyme, or combinations thereof, into the tissue of an organism to treat a deep tissue infection.

It is also an object of the invention to administer at least one holin lytic enzyme, at least one chimeric lytic enzyme, at least one shuffled lytic enzyme, or combinations thereof, intravenously.

In one embodiment of the invention, the treatments of a variety of illnesses caused by Streptococcus fasciae, and Staphylococcus aureus are disclosed.

In yet another embodiment of the invention, lysostaphin, the enzyme which lyses Staphylococcus aureus, can be included in the therapeutic agent.

In a further embodiment of the invention, conventional antibiotics may be included in the therapeutic agent with at least one holin lytic enzyme, at least one chimeric lytic enzyme, at least one shuffled lytic enzyme, holin lytic enzyme, or combinations thereof, with or without the presence of lysostaphin.

In another embodiment of the invention, more than one chimeric lytic enzyme, shuffled lytic enzyme, holin lytic enzyme, or combinations thereof, may be included in the therapeutic agent.

The therapeutic agent may be given parenterally, by means of an intramuscular, intradernal, or subcutaneous injection, or the agent may be given intravenously.

The present invention discloses the use of a variety of bacterial phage associated holin lytic enzymes, shuffled lytic enzymes, chimeric lytic enzymes, or combinations thereof, for the treatment of a wide variety of illnesses caused by bacterial infections. More specifically, the present invention discloses the parenteral application of a bacterial lytic enzyme, wherein the phage associated lytic enzyme is administered intramuscularly, subdermally, subcutaneously, or intravenously to treat a bacterial infection.

In another preferred embodiment of the invention, at least one lytic enzyme which is not selected from the group consisting of at least one shuffled lytic enzyme, chimeric lytic enzyme, and holin lytic enzyme may be used, in addition to the other enzymes.

### BRIEF DESCRIPTION OF THE DRAWING

FIG. 1 is an electron micrograph of group A streptococci treated with lysin showing the formation of a hole in the cell wall causing the cytoplasmic membrane to extrude and the cell contents to pour out.

### DETAILED DESCRIPTION OF THE INVENTION

The method for treating systemic or tissue bacterial infections comprises parenterally treating the infection with a therapeutic agent comprising an effective amount of at least one holin lytic enzyme, at least one chimeric lytic enzyme, at least one shuffled lytic enzyme, or combinations thereof, produced by a bacteria infected with a bacteriophage specific for the bacteria, and an appropriate carrier.

The method for treating bacterial infections comprises treating the infection with a therapeutic agent comprising an effective amount of a modified version of at least one lytic enzyme produced by a bacteria infected with a bacteriophage specific for the bacteria wherein the modified version of the one lytic enzyme is selected from the group consisting of shuffled lytic enzymes, chimeric lytic enzymes, and combinations thereof. The lytic enzyme is preferably in an environment having a pH which allows for activity of said lytic enzyme. A holin enzyme may be used alone or in conjunction with the administration of the modified lytic enzyme. The holin enzyme may be in its “natural” state, and may be a shuffled holin enzyme or may be a chimeric lytic enzyme.

The shuffled and chimeric enzymes may be produced either enzymatically or through recombinant DNA means. Any method may be used to produce these enzymes.

The lytic enzyme can be used for the treatment or prevention of Hemophilus influenza, Pseudomonas, Streptococcus pneumoniae, Streptococcus fasciae, Streptococcus group B, Listeria, Salmonella, E. coli, Campylobacter, and other bacteria, and any combination thereof. This lytic enzyme may be either supplemented by chimeric and/or shuffled lytic enzymes, or may be itself a chimeric and/or shuffled lytic enzyme. Similarly, a holin enzyme may be included, which may also be a chimeric and/or shuffled lytic enzyme.

A number of different bacteria may be treated. Among the bacteria which most often infect deep tissues, and, more specifically connective tissues, are Group A Streptococcus, Staphylococcus, Pseudomonas, and Clostridium. More than one lytic enzyme, and more than holin lytic enzyme, shuffled lytic enzyme, chimeric lytic enzyme, or combinations thereof, may be introduced into the infected body at a time.

A number of different methods may be used to introduce the lytic enzyme(s). These methods include introducing the modified lytic enzyme intravenously, intramuscularly, subcutaneously, and subdermally.

In one preferred embodiment of the invention, a deep tissue infection may be treated by injecting into the infected tissue of the patient a therapeutic agent comprising the appropriate holin lytic enzyme, chimeric lytic enzyme, shuffled lytic enzyme(s), lytic enzyme, or combinations thereof, and a carrier for the enzyme. The lytic enzymes used may be either supplemented by chimeric and/or shuffled lytic enzymes, or may be itself a chimeric and/or shuffled lytic enzyme. Similarly, a holin enzyme may be included, which may also be a chimeric and/or shuffled lytic enzyme. The carrier may be comprised of distilled water, a saline solution, albumin, a serum, or any combinations thereof. More specifically, solutions for infusion or injection may be prepared in a conventional manner, e.g. with the addition of preservatives such as p-hydroxybenzoates or stabilizers such as alkali metal salts of ethylene-diamine tetraacetic acid, which may then be transferred into fusion vessels, injection vials or ampules. Alternatively, the compound for injection may be lyophilized either with or without the other ingredients and be solubilized in a buffered solution or distilled water, as appropriate, at the time of use. Non-aqueous vehicles such as fixed oils and ethyl oleate are also useful herein.

In cases where intramuscular injection is the chosen mode of administration, an isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation. The pharmaceutical preparations according to the present invention are provided sterile and pyrogen free.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; glycine; amino acids such as glutamic acid, aspartic acid, histidine, or arginine; monosaccharides, disaccharides, and other carbohydrates including cellulose or its derivatives, glucose, mannose, trehalose, or dextrins; chelating agents such as EDTA; sugar alcohols such as mannitol or sorbitol; counter-ions such as sodium; non-ionic surfactants such as polysorbates, poloxamers, or polyethylene glycol (PEG); and/or neutral salts, e.g., NaCl, KCl, MgCl.sub.2, CaCl.sub.2, etc.

Glycerin or glycerol (1,2,3-propanetriol) is commercially available for pharmaceutical use. It may be diluted in sterile water for injection, or sodium chloride injection, or other pharmaceutically acceptable aqueous injection fluid, and used in concentrations of 0.1 to 100% (v/v), preferably 1.0 to 50% more preferably about 20%.

DMSO, which is an aprotic solvent with a remarkable ability to enhance penetration of many locally applied drugs. DMSO may be diluted in sterile water for injection, or sodium chloride injection, or other pharmaceutically acceptable aqueous injection fluid, and used in concentrations of 0.1 to 100% (v/v).

The carrier vehicle may also include Ringer's solution, a buffered solution, and dextrose solution, particularly when an intravenous solution is prepared.

Prior to, or at the time the lytic enzyme(s), including the modified lytic enzymes, is (are) put in the carrier system or oral delivery mode, it is preferred that the enzyme be in a stabilizing buffer environment for maintaining a pH range between about 4.0 and about 9.0, more preferably between about 5.5 and about 7.5 and most preferably at about 6.1. This is pH range is most suitable for the lysin enzyme for Streptococcus.

The stabilizing buffer should allow for the optimum activity of the modified lytic enzyme. The buffer may be a reducing reagent, such as dithiothreitol. The stabilizing buffer may also be or include a metal chelating reagent, such as ethylenediaminetetracetic acid disodium salt, or it may also contain a phosphate or citrate-phosphate buffer. The buffers found in the carrier can serve to stabilize the environment for the modified lytic enzymes.

The effective dosage rates or amounts of the modified lytic enzymes to treat the infection, and the duration of treatment will depend in part on the seriousness of the infection, the duration of exposure of the recipient to the infectious bacteria, the number of square centimeters of skin or tissue which are infected, the depth of the infection, the seriousness of the infection, and a variety of a number of other variables. The composition may be applied anywhere from once to several times a day, and may be applied for a short or long term period. The usage may last for days or weeks. Any dosage form employed should provide for a minimum number of units for a minimum amount of time. The concentration of the active units of enzyme believed to provide for an effective amount or dosage of enzyme may be in the range of about 100 units/ml to about 500,000 units/ml of composition, preferably in the range of about 1,000 units/ml to about 100,000 units/ml, and most preferably from about 10,000 to 100,000 units/ml. The amount of active units per ml and the duration of time of exposure depends on the nature of infection, and the amount of contact the carrier allows the modified lytic enzyme(s) to have. It is to be remembered that the modified lytic enzyme(s) works best when in a fluid environment. Hence, effectiveness of the modified lytic enzymes(s) in part related to the amount of moisture trapped by the carrier. For the treatment of septicemia, there should be a continuous intravenous flow of therapeutic agent into the blood stream. The concentration of modified lytic enzyme(s) for the treatment of septicemia is dependent upon the seriousness of the infection.

In order to accelerate treatment of the infection, the therapeutic agent may further include at least one complementary agent which can also potentiate the bactericidal activity of the modified lytic enzyme(s). The complementary agent can be penicillin, synthetic penicillins bacitracin, methicillin, cephalosporin, polymyxin, cefaclor. Cefadroxil, cefamandole nafate, cefazolin, cefixime, cefmetazole, cefonioid, cefoperazone, ceforanide, cefotanme, cefotaxime, cefotetan, cefoxitin, cefpodoxime proxetil, ceftazidime, ceftizoxime, ceftriaxone, cefriaxone moxalactam, cefuroxime, cephalexin, cephalosporin C, cephalosporin C sodium salt, cephalothin, cephalothin sodium salt, cephapirin, cephradine, cefuroximeaxetil, dihydratecephalothin, moxalactam, loracarbef. mafate, chelating agents and any combinations thereof in amounts which are effective to synergistically enhance the therapeutic effect of the modified lytic enzyme(s).

Additionally, the therapeutic agent may further comprise the enzyme lysostaphin for the treatment of any Staphylococcus aureus bacteria. Mucolytic peptides, such as lysostaphin, have been suggested to be efficacious in the treatment of S. aureus infections of humans (Schaffner et al., Yale J. Biol. & Med., 39:230 (1967) and bovine mastitis caused by S. aureus (Sears et al., J. Dairy Science, 71 (Suppl. 1): 244(1988)). Lysostaphin, a gene product of Staphylococcus simulans, exerts a bacteriostatic and bactericidal effect upon S. aureus by enzymatically degrading the polyglycine cross-link of the cell wall (Browder et al., Res. Comm., 19: 393-400 (1965)). U.S. Pat. No. 3,278,378 describes fermentation methods for producing lysostaphin from culture media of S. staphylolyticus, later renamed S. simulans. Other methods for producing lysostaphin are further described in U.S. Pat. Nos. 3,398,056 and 3,594,284. The gene for lysostaphin has subsequently been cloned and sequenced (Recsei et al., Proc. Natl. Acad. Sci. USA, 84: 1127-1131 (1987)). The recombinant mucolytic bactericidal protein, such as r-lysostaphin, can potentially circumvent problems associated with current antibiotic therapy because of its targeted specificity, low toxicity and possible reduction of biologically active residues. Furthermore, lysostaphin is also active against non-dividing cells, while most antibiotics require actively dividing cells to mediate their effects (Dixon et al., Yale J. Biology and Medicine, 41: 62-68 (1968)). Lysostaphin, in combination with the modified lytic enzyme(s) can be used in the presence or absence of the listed antibiotics. There is a degree of added importance in using both lysostaphin and the lysin enzyme in the same therapeutic agent. Frequently, when a body has a bacterial infection, the infection by one genus of bacteria weakens the body or changes the bacterial flora of the body, allowing other potentially pathogenic bacteria to infect the body. One of the bacteria that sometimes co-infects a body is Staphylococcus aureus. Many strains of Staphylococcus aureus produce penicillinase, such that Staphylococcus, Streptococcus, and other gram positive bacterial strains will not be killed by standard antibiotics. Consequently, the use of the modified lytic enzyme(s) and lysostaphin, possibly in combination with antibiotics, can serve as the most rapid and effective treatment of bacterial infections. In yet another preferred embodiment, the invention may include mutanolysin, and lysozyme.

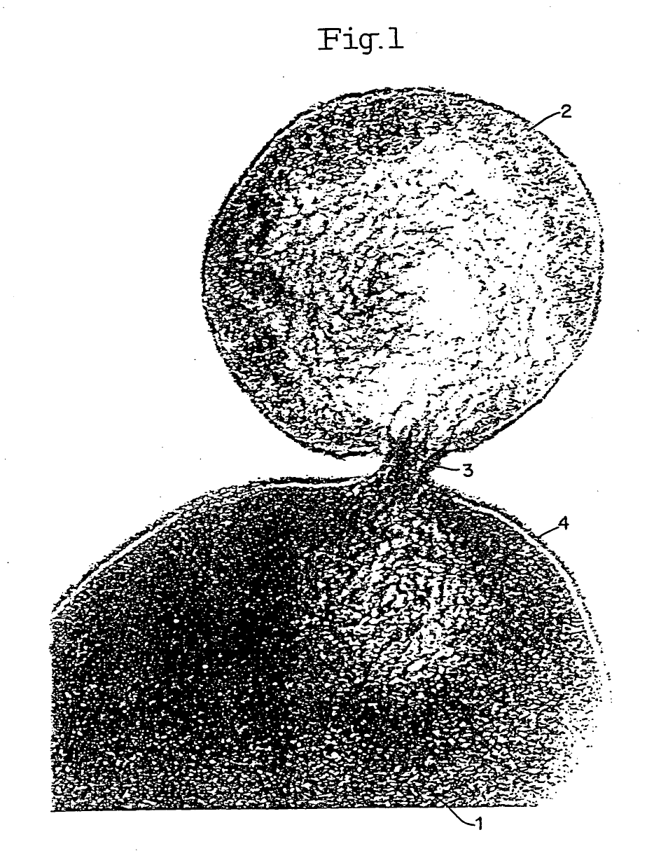

The use of lytic enzymes, including but not limited to holin lytic enzymes, chimeric lytic enzymes, shuffled lytic enzymes, and combinations thereof, rapidly lyse the bacterial cell. The thin section electron micrograph of FIG. 1 shows the results of a group A streptococci 1 treated for 15 seconds with lysin. The micrograph (25,000× magnification) shows the cell contents 2 pouring out through a hole 3 created in the cell wall 4 by the lysin enzyme.

As noted above, the use of the holin lytic enzyme, the chimeric lytic enzyme, and the shuffled lytic enzyme, may be accompanied by the use of a “natural” lytic enzyme, which has not been modified by the methods cited in U.S. Pat. No. 6,132,970, or by similar state of the art methods.

Many modifications and variations of the present invention are possible in light of the above teachings. It is, therefore, to be understood within the scope of the appended claims the invention may be protected otherwise than as specifically described.

What we claim is:

1) A method for the treatment of bacterial infections, comprising:
administering parenterally an effective amount of a therapeutic agent, said therapeutic agent comprising:
at least one lytic enzyme produced by a bacteria infected with a bacteriophage specific for said bacteria, wherein said at least one lytic enzyme is selected from the group consisting of shuffled lytic enzymes, chimeric lytic enzymes, holin lytic enzymes, and combinations thereof and;
a carrier for delivering said at least one lytic enzyme to the site of the infection.

2) The method according to claim 1, wherein said at least one lytic enzyme is for the treatment of Pseudomonas.

3) The method according to claim 1, wherein said at least one lytic enzyme is for the treatment of Streptococcus

4) The method according to claim 1, wherein said at least one lytic enzyme is for the treatment of Staphylococcus.

5) The method according to claim 1, wherein said at least one lytic enzyme is for the treatment of Clostridium.

6) The method according to claim 1, wherein said further therapeutic agent comprises a buffer that maintains pH of the composition at a range between about 4.0 and about 9.0.

7) The method according to claim 6, wherein the buffer maintains the pH of the therapeutic agent at the range between about 5.5 and about 7.5.

8) The method according to claim 6, wherein said buffer comprises a reducing reagent.

9) The method according to claim 8, wherein said reducing reagent is dithiothreitol.

10) The method according to claim 6, wherein said buffer comprises a metal chelating reagent.

11) The method according to claim 10, wherein said metal chelating reagent is ethylenediaminetetracetic disodium salt.

12) The method according to claim 6, wherein said buffer is a citrate-phosphate buffer.

13) The method according to claim 1, further comprising a bactericidal or bacteriostatic agent as a preservative.

14) The method according to claim 1, wherein said at least one lytic enzyme is lyophilized.

15) The method according claim 1, further comprising administering a concentration of about 100 to about 500,000 active enzyme units per milliliter of fluid in the wet environment of the nasal or oral passages.

16) The method according to claim 15, further comprising administering the concentration of about 100 to about 10,000 active enzyme units per milliliter of fluid in the wet environment of the nasal or oral passages.

17) The method according to claim 1, wherein said therapeutic agent is administered intravenously.

18) The method according to claim 1, wherein said therapeutic agent is administered intramuscularly.

19) The method according to claim 1, wherein said therapeutic agent is administered subcutaneously.

20) The method according to claim 1, wherein the therapeutic agent further comprises at least one complementary agent which potentiates the bactericidal activity of the at least one enzyme, said complementary agent being selected from the group consisting of penicillin, synthetic penicillins bacitracin, methicillin, cephalosporin, polymyxin, cefaclor. Cefadroxil, cefamandole nafate, cefazolin, cefixime, cefmetazole, cefonioid, cefoperazone, ceforanide, cefotanme, cefotaxime, cefotetan, cefoxitin, cefpodoxime proxetil, ceftazidime, ceftizoxime, ceftriaxone, cefriaxone moxalactam, cefuroxime, cephalexin, cephalosporin C, cephalosporin C sodium salt, cephalothin, cephalothin sodium salt, cephapirin, cephradine, cefuroximeaxetil, dihydratecephalothin, moxalactam, loracarbef. mafate and chelating agents in an amount effective to synergistically enhance the therapeutic effect of the at least one lytic enzyme.

21) The method according to claim 1, wherein said carrier comprises of distilled water, a saline solution, albumin, a serum, and any combinations thereof.

22) The method according to claim 1, wherein said carrier further comprises preservatives.

23) The method according to claim 22, wherein said preservatives comprise p-hydroxybenzoates.

24) The method according to claim 1, wherein said carrier comprises an isotonic solution for an injection, said isotonic solution comprising a compound selected from group consisting of sodium chloride, dextrose, mannitol, sorbitol, lactose, phosphate buffered saline, gelatin, albumin, a vasoconstriction agent and combinations

25) The method according to claim 24, wherein said further carrier further comprises DMSO.

26) The method according to claim 1, wherein said method is for the prophylactic treatment of infections.

27) The method according to claim 1, wherein said method is for the therapeutic treatment of infections.

28) The method according to claim 1, wherein said at least one said holin lytic enzyme is a shuffled holin lytic enzyme.

29) The method according to claim 1, wherein said at least one holin enzyme is a chimeric holin lytic enzyme.

30) The method according to claim 1, further comprising at least one lytic enzyme which is not selected from the group consisting of at least one shuffled lytic enzyme, chimeric lytic enzyme, and holin lytic enzyme.

31) A composition for the treatment of bacterial infections, comprising:
a therapeutic agent comprising:
an effective amount of at least one lytic enzyme produced by a bacteria infected with a bacteriophage specific for said bacteria, wherein said at least one lytic enzyme is selected from the group consisting of shuffled lytic enzymes, chimeric lytic enzymes, holin lytic enzymes, and combinations thereof, said at least one lytic enzyme having the ability to digest a cell wall of a specific said bacteria; and,
a carrier for the parenteral delivery of said at least one lytic enzyme to the site of the infection.

32) The composition according to claim 31, wherein the at least one lytic enzyme is for the treatment of Pseudomonas.

33) The composition according to claim 31, wherein the at least one lytic enzyme is for the treatment of Streptococcus

34) The composition according to claim 31, wherein the at least one lytic enzyme is for the treatment of Staphylococcus.

35) The composition according to claim 31, wherein the at least one lytic enzyme is for the treatment of Clostridium.

36) The composition according to claim 31, wherein said composition further comprises a buffer that maintains pH of the composition at a range between about 4.0 and about 9.0.

37) The composition according to claim 36, wherein the buffer maintains the pH of the composition at the range between about 5.5 and about 7.5.

38) The composition according to claim 36, wherein said buffer comprises a reducing reagent.

39) The composition according to claim 38, wherein said reducing reagent is dithiothreitol.

40) The composition according to claim 36, wherein said buffer comprises a metal chelating reagent.

41) The composition according to claim 40, wherein said metal chelating reagent is ethylenediaminetetracetic disodium salt.

42) The composition according to claim 36, wherein said buffer is a citrate-phosphate buffer.

43) The composition according to claim 31, further comprising a bactericidal or bacteriostatic agent as a preservative.

44) The composition according to claim 31, wherein said at least one lytic enzyme is lyophilized.

45) The composition according claim 31, further comprising administering a concentration of about 100 to about 500,000 active enzyme units per milliliter of fluid in the wet environment of the nasal or oral passages.

46) The composition according to claim 31, further comprising administering the concentration of about 1000 to about 100,000 active enzyme units per milliliter of fluid in the wet environment of the nasal or oral passages.

47) The composition according to claim 31, wherein said therapeutic agent is administered intravenously.

48) The composition according to claim 31, wherein said therapeutic agent is administered intramuscularly.

49) The method according to claim 31, wherein said therapeutic agent is administered subcutaneously.

50) The composition according to claim 31, wherein the therapeutic agent further comprises at least one complementary agent which potentiates the bactericidal activity of the lysine enzyme, said complementary agent being selected from the group consisting of penicillin, synthetic penicillins bacitracin, methicillin, cephalosporin, polymyxin, cefaclor. Cefadroxil, cefamandole nafate, cefazolin, cefixime, cefmetazole, cefonioid, cefoperazone, ceforanide, cefotanme, cefotaxime, cefotetan, cefoxitin, cefpodoxime proxetil, ceftazidime, ceftizoxime, ceftriaxone, cefriaxone moxalactam, cefuroxime, cephalexin, cephalosporin C, cephalosporin C sodium salt, cephalothin, cephalothin sodium salt, cephapirin, cephradine, cefuroximeaxetil, dihydratecephalothin, moxalactam, loracarbef. mafate and chelating agents in an amount effective to synergistically enhance the therapeutic effect of the lysin enzyme.

51) The composition according to claim 31, wherein said carrier is selected from the group consisting of distilled water, a saline solution, albumin, a serum, and any combinations thereof.

52) The composition according to claim 31, wherein said carrier further comprises preservatives.

53) The composition according to claim 52, wherein said preservatives comprise p-hydroxybenzoates.

54) The composition according to claim 31, wherein said carrier comprises an isotonic solution for an injection, said isotonic solution comprising a compound selected from group consisting of sodium chloride, dextrose, mannitol, sorbitol, lactose, phosphate buffered saline, gelatin, albumin, a vasoconstriction agent and combinations thereof.

55) The composition according to claim 31, wherein said carrier further comprises DMSO.

56) The method according to claim 31, wherein said method is for the therapeutic treatment of infections.

57) The method according to claim 31, wherein said at least one said holin lytic enzyme is a shuffled holin lytic enzyme.

58) The method according to claim 31, wherein said at least one holin enzyme is a chimeric holin lytic enzyme.

59) The method according to claim 31, further comprising at least one lytic enzyme which is not selected from the group consisting of at least one shuffled lytic enzyme, chimeric lytic enzyme, and holin lytic enzyme.
